(19) United States
(12) Patent Application Publication
(10) Pub. No.: US 2021/0030578 A1
(43) Pub. Date: Feb. 4, 2021

(54) ADJUSTABLE ORTHOTIC SHELLS

(71) Applicant: Scott Wimberley, Everett, WA (US)

(72) Inventor: Scott Wimberley, Everett, WA (US)

(21) Appl. No.: 16/942,319

(22) Filed: Jul. 29, 2020

Related U.S. Application Data
(60) Provisional application No. 62/881,470, filed on Aug. 1, 2019.

(51) Int. Cl. A61F 5/058; A43B 7/00

(52) U.S. Cl. CPC A61F 5/0585; A61F 2005/0197; A43B 7/00

(57) ABSTRACT

Apparatus and method for an adjustable orthotic. In one embodiment, an adjustable orthotic may include: a calf shell with a first anchor plate configured to receive a calf of a user; a foot shell with a second anchor plate configured to receive a foot of the user; and a removeable spring comprising a top, a bottom, and a recurved shape, wherein the top of the spring connects to the calf shell by the first anchor plate and the bottom of the spring connects to the foot shell by the second anchor plate. In one embodiment, an apparatus may include: a first anchor plate comprising one or more first raised threaded bosses; a second anchor plate comprising one or more second raised threaded bosses; a spring, wherein a top of the spring connects to the first anchor plate with one or more first fasteners and a bottom of the spring connects to the second anchor plate with one or more second fasteners; a first negative space located above the first anchor plate for alignment of the top of the spring with the first anchor plate; a second negative space located above the second anchor plate for alignment of the bottom of the spring with the second anchor plate; and a negative space tooling comprising one or more alignment openings, wherein the one or more alignment openings align with the one or more first raised threaded bosses and the one or more second raised threaded bosses. A method may include: attaching an anchor plate to a forming structure; attaching a first fastener to the anchor plate; applying one or more sheets over the first fastener and onto the anchor plate; compressing the one or more sheets onto the anchor plate using a compactor; attaching a negative space tooling to the anchor plate; forming a shell over the forming structure, the anchor plate, and the one or more sheets, wherein forming the shell integrates the anchor plate and the one or more sheets into the shell; removing a portion of the shell exposing the negative space tool; and removing the negative space tooling wherein exposing a top surface of the anchor plate, wherein the top surface of the anchor plate includes one or more openings configured to receive one or more second fasteners.

### CROSS REFERENCE TO RELATED APPLICATIONS

This application claims priority to U.S. Provisional Application, Ser. No. 62/881,470, filed on Aug. 1, 2019, which is hereby incorporated by reference for all purposes.

### BACKGROUND

A variety of medical conditions exist that inhibit individuals from having full range of motion at the ankle as well as pain upon weight bearing. This inability to move and/or being in pain keeps the individual unable to move in a normal way. This altered motion requires higher levels of energy from the individual for common tasks and for higher activity levels, such as running. These energy losses and pain may make the activity impossible. As an example, severe ankle or foot crush injuries regularly require surgeries and internal metal fixation hardware to be permanently implanted. This hardware inhibits motion and regularly causes pain if forced to move. Treatment and management for this individual requires an “off-loading” style brace to prevent force being transferred through the ankle as well as a dynamic factor to help with energy losses and provide energy return. Common designs for this type of brace utilize custom formed composite shells to properly position and support the individual's lower limb that incorporates a composite spring to support the individual's weight and off-load the ankle or foot.

### BRIEF DESCRIPTION OF THE DRAWINGS

The present description will be understood more fully from the detailed description given below and from the accompanying drawings of various embodiments of the present embodiment, which is not to be taken to limit the present embodiment to the specific embodiments but are for explanation and understanding.

FIG. 1A illustrates a side perspective cross sectional view of an adjustable orthotic, according to an embodiment.

FIG. 1B illustrates a back perspective view of the spring in FIG. 1A, according to an embodiment.

FIG. 2A illustrates the anchor plate with raised threaded bosses and introducer tools in FIG. 1A, according to an embodiment.

FIG. 2B illustrates the anchor plate with raised threaded bosses and a compactor tool, according to an embodiment.

FIG. 2C illustrates the anchor plate with the compactor tool pressed around the introducer tools, over the top of the first raised threaded boss and the second raised threaded boss, and onto the base portion, according to an embodiment.

FIG. 2D illustrates a cross-sectional view of the anchor plate with the compactor tool pressed around the introducer tools, over the top of the first raised threaded boss and the second raised threaded boss, and onto the base portion, according to an embodiment.

FIG. 3A illustrates a portion of the calf shell or the foot shell and a negative space tooling, according to an embodiment.

FIG. 3B illustrates a side perspective view of the portion of the calf shell or the foot shell and the negative space tooling, according to an embodiment.

FIG. 4 illustrates a flowchart of a method for integrating the anchor plate into the calf shell or the foot shell of FIG. 1A, according to an embodiment.

### DETAILED DESCRIPTION

The disclosed adjustable orthotics will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations, however, for the sake of brevity, each and every contemplated variation is not individually described in the following detailed description.

Throughout the following detailed description, a variety of adjustable orthotic examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

Some medical conditions may inhibit an individual from moving their body parts in a normal way. For example, neuropathic condition sufferers, such as cerebral palsy sufferers, exhibiting spastic diplegia lose their ability to walk normally. The neuropathic conditions may be chronic neuromuscular conditions of hypertonia and spasticity that cause tightness or stiffness in the muscles of an individual's lower extremities, such as their legs, hips, and pelvis.

Conventionally, to increase the ability an individual with the neuropathic condition to walk, the individual may undergo gait analysis so that medical personnel treating the condition can determine the best assistive devices for them. Depending on the severity of the neuropathic condition, the medical personnel may prescribe a walker or crutches to aid the individual in walking or moving around. The walkers or crutches may increase the individual's ability to remain stable while walking or moving. However, the walkers or crutches may be difficult to transport and may also limit the individual's ability to move freely while not drawing attention to themselves.

To aid the individual to increase or maintain mobility, the medical personnel may prescribe orthotics to reduce or prevent undesirable movement of body parts that may cause pain or instability in the individual's movements. For example, orthotics may be employed to enable an individual to hold a limb in advantageous positions to improve the functionality of the limb. However, while each individual's physiology and body characteristics are different, conventional orthotics fail to take into account the individual's physiology and body characteristics. The conventional orthotics may therefore not properly match the individual's physiology and body characteristics and cause other undesirable movements by the body parts they are attached to and/or cause discomfort to the individual using the orthotics. For example, when the orthotics do not match individual's physiology and body characteristics, the orthotic may reduce one undesirable movement caused by the neuropathic condition while introducing a new undesirable movement as the individual walks or moves. The proper balance of spring force can provide a needed support and immobilization while still offering a dynamic flex response to loading. Attaching a dynamic or specialized spring to the composite shells compensates for the high levels of force generated during general ambulation and/or running.

Implementations of the disclosure addresses the above-mentioned deficiencies and other deficiencies by providing methods, systems, devices, and/or apparatuses to decrease undesirable movements by an individual with neuropathic movements and increase their mobility. An adjustable orthotic may include a foot shell, a spring, and a calf shell. The spring may be connected to the calf shell by a first anchor and a first fastener and may be connected to the foot shell by a second anchor and a second fastener. The first anchor may be integrated into the calf shell and the second anchor may be integrated into the foot shell. An advantage of an adjustable orthotic is that the orthotic may be customized or individualized to the user by allowing a variety of springs to be connected to the foot shell and the calf shell. In another example of an adjustable orthotic, the foot shell is replaced with a prosthetic device.

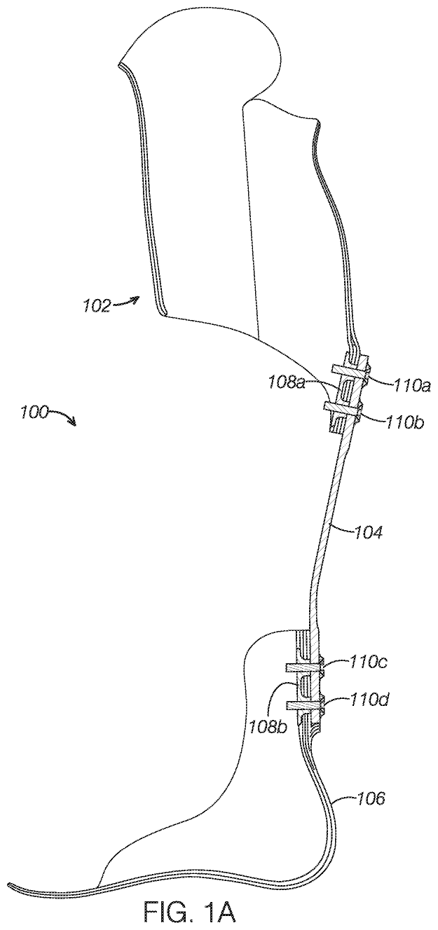

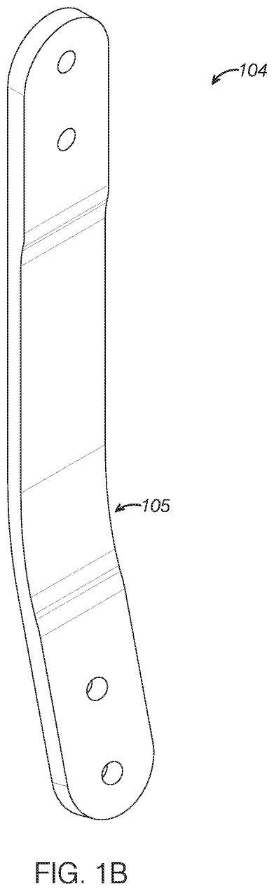

FIG. 1A illustrates a side perspective cross-sectional view of an adjustable orthotic 100, according to an embodiment. The adjustable orthotic 100 may include a calf shell 102, a spring 104, a foot shell 106, a first anchor plate 108a, and a second anchor plate 108b. In one embodiment, the calf shell 102 may be shaped to conform to a calf of the user. In one example, the calf shell 102 may include a strap to secure the calf shell 102 to the calf of the user.

The top of the spring 104 may be connected to a bottom of the calf shell 102 with the first anchor plate 108a. The spring 104 may extend perpendicularly downwardly toward the foot shell 106. The foot shell 106 may be shaped to conform to at least a portion of the foot of the user. In one example, a bottom of the spring 104 may connect a top of the foot shell 106 with the second anchor plate 108b. In one example, the foot shell 106 may conform around an ankle and a bottom of the foot of the user. In another example, the adjustable orthotic 100 may provide a user with an interface or bridge for anatomical segments, such as replacing the foot shell 106 with prosthetic componentry.

In one embodiment, the spring 104 may be interchangeable with other springs with various characteristics to adjust or tune the adjustable orthotic 100 based on physiological and body characteristics of the user wearing the adjustable orthotic 100. For example, different springs 104 may have different characteristics to customize the adjustable orthotic 100 to the user. The spring characteristics may include a length of the spring, a thickness of the spring, a width of the spring, a flexibility or a rigidity of the spring, a material of the spring, and so forth. The user and/or a medical personnel may connect different springs with various characteristics to the calf shell 102 and the foot shell 106 to customize the adjustable orthotic 100 to the user. For example, when the user is relatively short, the medical personnel may use a spring 104 that is relatively short in length to adjust the adjustable orthotic 100 to the height of the user. In another example, when the user is relatively light in body weight, the medical personnel may use a spring 104 with low flexibility to reduce an amount of rebound force by the spring 104 as the user walks or runs. In another embodiment, the medical personnel may select the spring 104 with given characteristics based on characteristics of the user, such as a height of the user, a weight of the user, an activity level of the user, a diagnosed condition of the user, and so forth. The interchangeable springs 104 may enable the user and/or the medical personnel to adjust or tune the adjustable orthotic 100 specifically to the user to optimize a performance of the adjustable orthotic 100 and reduce undesirable movements by the user.

The size, shape, and dimensions of the calf shell 102, the spring 104, the foot shell 106, the anchor plates 108a and 108b, and/or the fasteners 110a-110d are not intended to be limiting and may vary based on the user. The materials of the calf shell 102, the spring 104, the foot shell 106, the anchor plates 108a and 108b, and/or the fasteners 110a-110d are not intended to be limiting and may vary based on the user.

FIG. 1B illustrates a back perspective view of the spring 104, according to an embodiment. As discussed above, the spring 104 of the adjustable orthotic brace 100 may be interchangeable to provide a modular system that allows for tuning a dynamic response and/or a static alignment of the adjustable orthotic brace 100 to provide optimum performance from the adjustable orthotic brace 100. The dynamic response may be adjusted through the selection and utilization of differing springs 104 with each having different stiffness by tuning each spring 104 through the reduction of thickness at 20% increments, resulting in as 20% difference in stiffnesses between each spring in a set of springs. Thus, the adjustable orthotic brace 100 may be configured to connect the calf brace 102 and the foot brace 106 together with springs 104 categorized by a number increasing levels of stiffness.

In one embodiment, a set of springs may have uniform dimensions with different stiffness levels to adjust the adjustable orthotic brace 100 for optimal stiffness while otherwise maintaining the same configuration of the adjustable orthotic brace 100. By maintaining the same configuration of the adjustable orthotic brace 100 except for the stiffness of the spring 104, a medical personnel may adjust the adjustable orthotic brace 100 to determine an optimal stiffness of the spring 104 for each user while keeping all other variables equal to provide a repeatable tuning test of the optimal spring stiffness for the user.

In one embodiment, the spring 104 may have a contoured shape where the top of the spring 104 extends downward along a vertical plane until it reaches an inflection point 105. At the inflection point 105, the spring 104 may curve away from a leg of the user at a defined angle. In one example, the spring 104 may curve rearward so that the spring 104 may extend from the leg or calf of the user to a heel of the user that is farther back than the plane the calf extends along or on a different plane or angle than the calf.

In another embodiment, the spring may be contoured at the inflection point 105 to provide a low profile that fits closely to a profile limb of the user or the negative shape of the limb, such as a leg of the user. In one example, the spring 104 may have a recurved “reverse C” design. The “reverse C” design may match the posterior profile of the limb to bring the spring 104 as close to the limb as possible to provide the least amount of space between the spring 104 and the limb. The “reverse C” design offers enhanced bending ease for plantar motions of the foot of the user and increased stiffness for dorsal motions. This enhanced planar bending absorbs excess energy generated at heel strike/collision in the gait cycle by loading the spring 104 through planar motion and reciprocally through dorsal motion unloading helps bring the limb through to midstance. From midstance the resistance to dorsal motion of the foot enables the spring 104 to offload the limb and provide any stored energy back to propel the body forward at toe off.

In another embodiment, the orientation or alignment of the spring 104 may be adjusted or changed relative to the calf brace 102 and/or the foot brace 106 of the adjustable orthotic brace 100 using one or more alignment shims (not shown) that are placed between the spring 104 and anchors 108a and/or 108b.

In one embodiment, the contoured design of the spring 104 may be configured to deform to absorb energy as the user walks with the heal portion of the foot and release the absorbed energy when the user's foot reaches a toe off position as they walk. The absorption and release of the energy by the spring 104 may reduce undesired movements by the leg or foot as the user walks and increase desired movements or control the desired movements of the leg or foot to aid the user in walking or other movements. In one embodiment, the spring 104 may aid the user through their movement of the gait cycle. As discussed above, the “reverse C” design may transfer a ground force reaction posterior to a body of the spring 104 at an initial point of loading response causing a planar flection moment around the spring 104 at a first stage of the gait cycle and as the user moves through mid-stance and into a loading response or stance, the spring 104 may deflect in an opposite direction and stores energy to be released at toe off. Thickened mounting shanks on a body of the spring 104 focus bend energy to a mid-body of the spring 104 and away from mounting hardware, such as the anchor plates 108a and 108b, and/or the fasteners 110a-110d. In another embodiment, the spring 104 has shouldered ends to focus bend in the body of the spring 104. In one example, the spring 104 comprises a two-hole mounting system with mounting openings clearance for angular fitment.

As discussed above, the spring 104 may be interchangeable with other springs. In one embodiment, the spring 104 may be interchangeable to adjust the adjustable orthotic brace 100 to the physiology and body characteristics of the user. In another embodiment, the spring 104 may be interchangeable to enable the user and/or the medical personnel to replace the spring 104 or other parts of the adjustable orthotic brace 100 if the spring 104 or other parts become broken or damaged. Springs 104 may be developed as a set with an even gradient of force.

In another embodiment, the spring 104, the first anchor 108, and/or the second anchor 110 may be a lightweight material, such as aluminum, plastic, polyurethane, and so forth. In another embodiment, the spring 104, the first anchor 108, and/or the second anchor 110 may be metal, fiberglass, glass, carbon, polycarbonate, carbon fiber, Kevlar®, thermoplastic and so forth. The calf brace 102 and/or the foot brace 106 may be a cast material, such as plaster, fiberglass, polyurethane, plastic, carbon, polycarbonate, carbon fiber, Kevlar®, thermoplastic, and so forth. In one embodiment, the spring 104 may be a carbon composite or other fiber reinforced plastic.

In one embodiment, the spring 104 may have a flat shape, a round shape, a curved shape, a pole shape, a cylinder shape, and so forth. In one example, the spring 104 has a flat bar shape with a single plane bend. The shape, contour, and/or position of the spring 104 for the adjustable orthotic brace 100 may vary. In another embodiment, the spring 104 may be cut to length by the user or a medical personnel to customize the spring to the physiological and body characteristics of the user and may be mounted to the calf brace 102 and the foot brace 106 after it is cut to length. In one example, the spring 104 may vary in length from 165 mm to 310 mm. In another example, the spring 104 may also vary in width from 18 mm to 35 mm. In one example, the spring 104 may vary in thickness from 2.5 mm to 12 mm. In the “reverse C” design, the spring 104 may have an angle range of 5 degrees to 20 degrees.

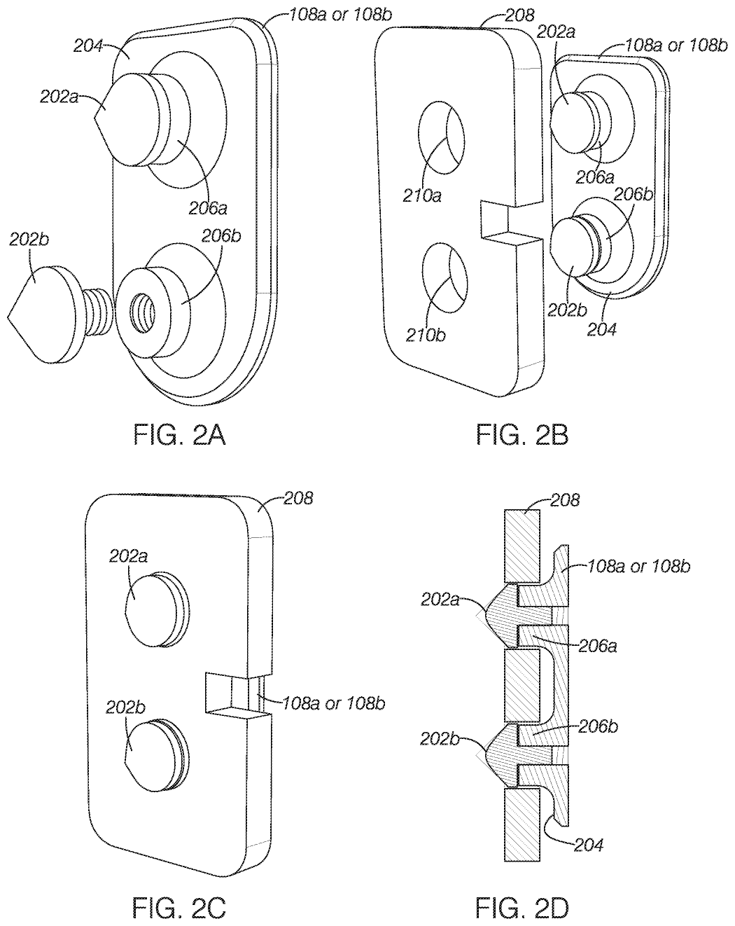

FIG. 2A illustrates the anchor plate 108a or 108b in FIG. 1A with raised threaded bosses 206a and 206b and introducer tools 202a and 202b. Some of the features in FIG. 2A are the same or similar to some of the features in FIG. 1A as noted by same reference numbers, unless expressly described otherwise. The anchor plate 108a or 108b may include a base portion 204 that is a flat sheet or disk that may extend along a plane. In one embodiment, the base portion 204 may be square shaped, oval shaped, rectangularly shaped, circularly shaped, or other shapes. In another embodiment, the base portion 204 may have square edges or rounded edges.

The anchor plate 108a or 108b may include a first raised threaded boss 206a and/or a second raised threaded boss 206b. The raised threaded bosses 206a and/or 206b may extend vertically from the base portion 204. In one embodiment, the first raised threaded boss 206a and/or the second raised threaded boss 206b may have a conical shape or volcanic shape with a flat top surface.

The first raised threaded boss 206a may be configured to receive a first introducer tool 202a and the second raised threaded boss 206b may be configured to receive a second introducer tool 202b. In one example, the raised threaded boss 206a and the second raised threaded boss 206b may prove a two-bolt pattern for a 25.4 mm bolt pattern. In another example, the first introducer tool 202a and/or the second introducer tool 202b may be bolts with a male threaded portion with the raised threaded boss 206a and the second raised threaded boss 206b having corresponding female threaded portions. In another example, the first introducer tool 202a and/or the second introducer tool 202b may have a flat top. In another example, the introducer tool 202a and/or the introducer tool 202b may have a pointed top. In one embodiment, the first introducer tool 202a and/or the second introducer tool 202b may be aluminum for weight savings or, in another embodiment, stainless steel for strength and corrosion resistance. In one embodiment, the pointed top may be shaped or configured to puncture or form holes in a sheet (such as a carbon fiber sheet) as the sheets are pressed onto the base portion 204, as discussed below. In one example, the sheets may be layered.

In one embodiment, the anchor plate 108a and/or 108b may withstand high and repeated force loads transferring through the spring 104 in FIG. 1A from the ground forces generated by the user. A mounting surface of the anchor plate 108a and/or 108b may be a flat space and/or a level space for mounting the spring 104. The anchor plate 108a and/or 108b may have a bolt pattern corresponding to the spring 104. In one embodiment, the anchor plate 108a and/or 108b may provide an increased thread engagement because the threaded first introducer tool 202a and/or the second introducer tool 202b pass through the sheets.

FIG. 2B illustrates the anchor plate 108a and 108b with raised threaded bosses 206a and 206b and a compactor tool 208, according to an embodiment. Some of the features in FIG. 2B are the same or similar to some of the features in FIGS. 1A and 2A as noted by same reference numbers, unless expressly described otherwise. The compactor tool 208 may be a flat plate that includes openings 210a and 210b that correspond with the introducer tools 202a and 202b to fit over the introducer tools 202a and 202b and the raised threaded boss 206a and the second raised threaded boss 206b, respectively. As discussed above, the pointed tops of the introducer tool 202a and/or the introducer tool 202b may be configured to puncture or form a hole in a sheet(s) as the sheet(s) are pressed around the raised threaded boss 206a and the second raised threaded boss 206b and against the base portion 204. When the sheet(s) is pressed onto the base portion 204, the compactor tool 208 may be pressed on top of the sheet(s) and the base portion 204 such that the sheet(s) may be compacted onto the base portion 204, the first threaded boss 206a, and the second threaded boss 206b. Pressing the sheet(s) onto the pointed tops of the first introducer tool 202a and/or the second introducer tool 202b may allow the first introducer tool 202a and/or the second introducer tool 202b to pass through the weaves of the sheet(s), leaving the sheet(s) spread around raised threaded boss 206a and the second raised threaded boss 206b pressed down against onto the base portion 204. In another embodiment, the compactor 208 may be used in indicating a length fasteners 10a-10d may be to attach the spring 104 to the calf shell 102 and the foot shell 106 in FIG. 1A. A proper length of the fasteners 110a-10d may provide a full thread engagement between the fasteners 10a-10d and the anchor plate 108a and/or 108b.

FIG. 2C illustrates the anchor plate 108a and/or 108b with the compactor tool 208 pressed around the top of the introducer tools 202a and 202b, over the top of the first raised threaded boss 206a and the second raised threaded boss 206b, and onto the base portion 204, according to an embodiment. Some of the features in FIG. 2C are the same or similar to some of the features in FIGS. 1A-2B as noted by same reference numbers, unless expressly described otherwise. In one example, when the compactor tool 208 is pressed over the top of the first raised threaded boss 206a and the second raised threaded boss 206b and onto the base portion 204, at least a portion of the first introducer tool 202a and the second introducer tool 202b may protrude or extend beyond a top surface of the compactor tool 208. The first introducer tool 202a and/or the second introducer tool 202b may provide a visual indicator when the sheet(s) have reached a defined thickness, such as a thickness of 6.7 millimeters (mm). The introducer tools 202a and 202b may also extend through the sheet(s) offering positive mechanical keying into the sheet structure.

FIG. 2D illustrates a cross-sectional view of the anchor plate 108a and/or 108b with the compactor tool 208 pressed around the introducer tools 202a and 202b, over the top of the first raised threaded boss 206a and the second raised threaded boss 206b, and onto the base portion 204, according to an embodiment. Some of the features in FIG. 2D are the same or similar to some of the features in FIGS. 1A-2C as noted by same reference numbers, unless expressly described otherwise. FIG. 2D further illustrates a portion of the first fastener 202a and the second fastener 202b which may protrude or extend beyond a top surface of the compactor tool 208.

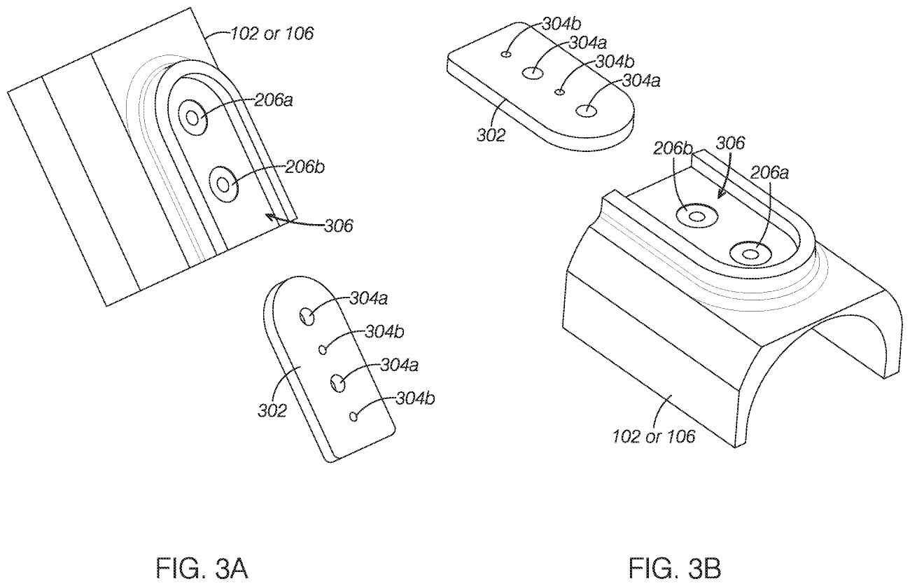

FIG. 3A illustrates a portion of the calf shell 102 or the foot shell 106 and a negative space tooling 302, according to an embodiment. Some of the features in FIG. 3A are the same or similar to some of the features in FIGS. 1A-2D as noted by same reference numbers, unless expressly described otherwise. The negative space tooling 302 may include alignment openings 304a which correspond with the raised threaded bosses 206a and/or 206b for orientation datum. In one example, the negative space tooling 302 may include weep openings 304b to allow for excess fluid to escape during the assembly process.

In one embodiment, when the anchor plate 108a and/or 108b is integrated into the calf shell 102 or the foot shell 106, the negative space tooling 302 may be placed over top of the anchor plate 108a and/or 108b and then a shell material (such as plaster, plastic, rubber, polyurethane, fiberglass, and so forth) may be formed over top of the anchor plate 108a and/or 108b and the negative space tooling 302. These integrated anchor plates 108a and 108b require no post processing assembly as the anchor plate 108a and 108b is integrated into the calf shell 102 or the foot shell 106.

When the shell material of the calf shell 102 or the foot shell 106 is formed, then a portion of the shell material may be removed to expose the negative space tooling 302. When the negative space tooling 302 is exposed, the negative space tooling 302 may be removed to expose a top surface of the first raised threaded boss 206a and the second raised threaded boss 206b, clearly delineating the anchor area. The negative space tooling 302 may ensure proper material compaction around first raised threaded boss 206a and the second raised threaded boss 206b. The top surfaces of the raised threaded bosses 206a and 206b may provide access to the female threads to enable mechanical fastening to anchor plate 108a and/or 108b.

The negative space tooling 302 may also create a cavity or negative space 306 for the spring 104 in FIGS. 1A-B to be inserted into when fastening the spring 104 to the calf shell 102 or the foot shell 106. In one example, negative space tooling 302 may be the same size and shape as an end portion of the spring 104, such that the cavity or negative space 306 may be a space that corresponds to the size and shape of the spring 104. Then the negative space 306 may be a flat, true surface in the correct plane of orientation for the spring 104 mounting position. When the spring 104 is inserted into the cavity or negative space 306, fasteners 110a-110d may connect the spring 104 to the female threads of the first raised threaded boss 206a and the second raised threaded boss 206b. The visible top of the raised threaded bosses 206a and 206b left from the negative space tooling 304 offer a visual safety check that all fasteners 110a-110d are positively being threaded into the anchor plate 108a ad/nor 108b.

FIG. 3B illustrates side perspective view of the portion of the calf shell 102 or the foot shell 106 and the negative space tooling 302, according to an embodiment. The negative space tooling 302 may orient or align the spring 104 with the calf shell 102 and/or the foot shell 106 when attached by keeping a mounting face of the spring 104 perpendicular to the internal thread of the first raised threaded boss 206a and the second raised threaded boss 206b and against face 306. The orientation or alignment may increase a reliability for a fitting of the adjustable orthotic 100 to a user. The negative space 306 may also create a radiused edge border around an end portion of the spring 104 to protect the spring from catching on a user's garments. An additional benefit of the negative space 306 is mechanical support provided to the spring 104, such that in the event of a hardware loosening or failure, walls created by the negative space 306 offer the mechanical support to the spring 104 by preventing side to side motions during use.

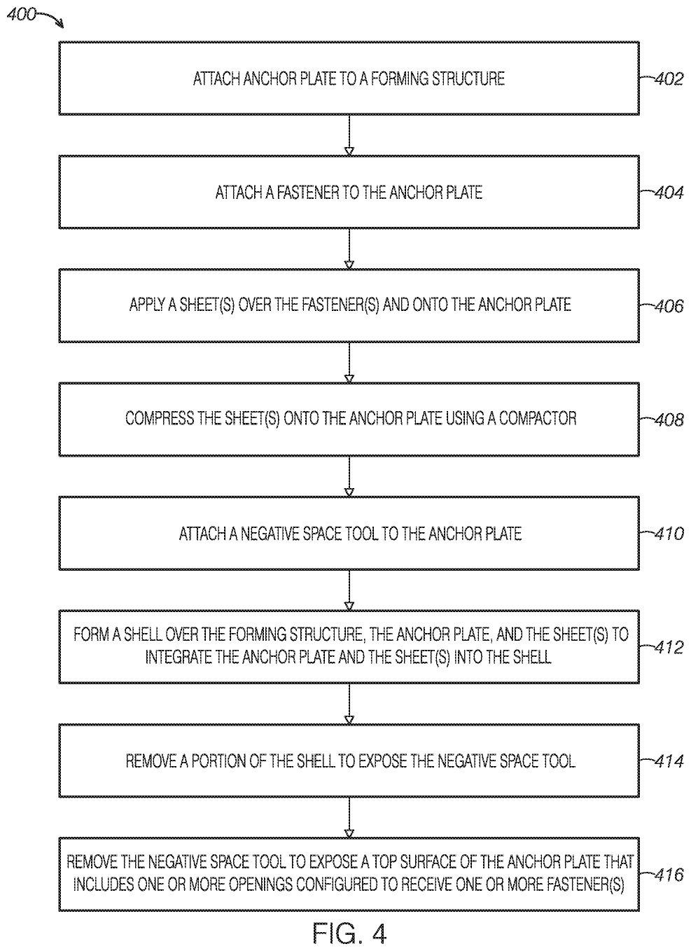

FIG. 4 illustrates a flowchart 400 of a method for integrating the anchor plate 108a and/or 108b into the calf shell 102 or the foot shell 106 of FIG. 1A, according to an embodiment. The method may include attaching an anchor plate to a forming structure (block 402). In one embodiment, the forming structure may be casted impression of a leg of the user. The method may include attaching the fastener(s) to the anchor plate (block 404). The method may include applying a sheet or sheets (such as carbon fiber sheets) over the introducer tool(s) and around the raised threaded bosses of the anchor plate (block 406). The method may include compressing the sheet(s) onto the anchor plate using a compacting tool (block 408). In one example, the compressing of the sheets onto the anchor plate may ensure all sheets around the anchor plate are compacted and uniformly compressed. This compaction around the anchor plate may occur several times as the sheets are attached to the anchor plate.

The method may include attaching a negative space tool to the anchor plate (block 410). For example, once the sheets are compacted onto the anchor plate, the introducer tools may be removed and the negative space tool may be attached to the anchor plate. In another example, fabrication materials may be added over the forming structure, the anchor plate, and the negative space tool to create the calf shell 102 and/or the foot shell 106.

The method may include forming a shell over the forming structure, the anchor plate and the sheet(s) into the shell (block 412). For example, the forming structure, the anchor plate, and the sheet(s) may be vacuum bagged and saturated with forming materials (such as epoxy resin). Once the forming materials have cured, the parts may be demolded and finished.

The method may include removing a portion of the shell to expose the negative space tool (block 414). For example, the user may use a grinder or knife to remove the one more layers of the calf shell or the foot shell. The method may include removing the negative space tool to expose a top surface of the anchor plate that includes one or more openings configured to receive one or more fasteners (block 416).

The disclosure above encompasses multiple distinct embodiments with independent utility. While these embodiments have been disclosed in a particular form, the specific embodiments disclosed and illustrated above are not to be considered in a limiting sense as numerous variations are possible. The subject matter of the embodiments includes the novel and non-obvious combinations and sub-combinations of the various elements, features, functions and/or properties disclosed above and inherent to those skilled in the art pertaining to such embodiments. Where the disclosure or subsequently filed claims recite “a” element, “a first” element, or any such equivalent term, the disclosure or claims is to be understood to incorporate one or more such elements, neither requiring nor excluding two or more such elements.

Applicant(s) reserves the right to submit claims directed to combinations and sub-combinations of the disclosed embodiments that are believed to be novel and non-obvious. Embodiments embodied in other combinations and sub-combinations of features, functions, elements and/or properties may be claimed through amendment of those claims or presentation of new claims in the present application or in a related application. Such amended or new claims, whether they are directed to the same embodiment or a different embodiment and whether they are different, broader, narrower or equal in scope to the original claims, are to be considered within the subject matter of the embodiments described herein.

1. An adjustable orthotic, comprising:
a calf shell with a first anchor plate configured to receive a calf of a user;
a foot shell with a second anchor plate configured to receive a foot of the user; and
a removeable spring comprising a top, a bottom, and a recurved shape, wherein the top of the spring connects to the calf shell by the first anchor plate and the bottom of the spring connects to the foot shell by the second anchor plate.

2. The adjustable orthotic of claim 1, wherein the recurved shape is a reverse C shape.

3. The adjustable orthotic of claim 1, wherein the removeable spring further comprises a thickness and wherein the thickness is configured to be reducible whereby enabling adjustment of a stiffness of the removeable spring.

4. The adjustable orthotic of claim 1, wherein the removeable spring further comprises an inflection point, and wherein the removeable spring curves away from the calf of the user at an angle at the inflection point.

5. The adjustable orthotic of claim 4, where in the angle ranges from five degrees to 20 degrees.

6. The adjustable orthotic of claim 1, wherein the top and the bottom of the removeable spring further comprises reinforced, thickened shoulders.

7. The adjustable orthotic of claim 1, wherein the removeable spring further comprises a two-hole mounting system.

8. The adjustable orthotic of claim 1, wherein the removeable spring further comprises an adjustable length.

9. The adjustable orthotic of claim 1, wherein the removeable spring is comprised of carbon composite.

10. An apparatus, comprising:
a first anchor plate comprising one or more first raised threaded bosses;
a second anchor plate comprising one or more second raised threaded bosses;
a spring, wherein a top of the spring connects to the first anchor plate with one or more first fasteners and a bottom of the spring connects to the second anchor plate with one or more second fasteners;
a first negative space located above the first anchor plate for alignment of the top of the spring with the first anchor plate;
a second negative space located above the second anchor plate for alignment of the bottom of the spring with the second anchor plate; and
a negative space tooling comprising one or more alignment openings, wherein the one or more alignment openings align with the one or more first raised threaded bosses and the one or more second raised threaded bosses.

11. The apparatus of claim 10, wherein the first anchor plate is integrated with a calf shell or a prosthetic.

12. The apparatus of claim 10, wherein the second anchor plate is integrated with a foot shell or a prosthetic.

13. The apparatus of claim 10, further comprising:
one or more sheets compressed onto the first anchor plate;
a calf shell formed over the first anchor plate, the one or more sheets, and the negative space tooling; and
wherein the negative space tooling is configured for creation of the first negative space once the negative space tooling is removed, exposing a first top surface of the one or more first raised threaded bosses for enabling alignment of the spring and the one or more first fasteners.

14. The apparatus of claim 10, further comprising:
one or more sheets compressed onto the second anchor plate;
a foot shell formed over the second anchor plate, the one or more sheets, and the negative space tooling; and
wherein the negative space tooling is configured for creation of the second negative space once the negative space tooling is removed, exposing a second top surface of the one or more second raised threaded bosses for enabling alignment of the spring and the one or more second fasteners.

15. A method, comprising:
attaching an anchor plate to a forming structure;
attaching a first fastener to the anchor plate;
applying one or more sheets over the first fastener and onto the anchor plate;
compressing the one or more sheets onto the anchor plate using a compactor;
attaching a negative space tooling to the anchor plate;
forming a shell over the forming structure, the anchor plate, and the one or more sheets, wherein forming the shell integrates the anchor plate and the one or more sheets into the shell;
removing a portion of the shell exposing the negative space tool; and
removing the negative space tooling wherein exposing a top surface of the anchor plate, wherein the top surface of the anchor plate includes one or more openings configured to receive one or more second fasteners.

16. The method of claim 15, wherein:
the anchor plate comprises a first raised threaded boss and a second raised threaded boss;
the first fastener comprises a first introducer tool configured to thread into the first raised threaded boss; and
the second fastener comprises a second introducer tool configured to thread into the second raised threaded boss.

17. The method of claim 16, further comprising:
compressing the one or more sheets over the first and second introducer tools and around the first and second raised threaded boss of the anchor plate.

18. The method of claim 15, wherein the shell is a calf shell.

19. The method of claim 15, wherein the shell is a foot shell.

20. The method of claim 15, further comprising:
repeating the compressing of the one or more sheets until the one or more sheets are uniformly compacted onto to the anchor plate.
